SEQUENCE LISTING (15 sequences)

SEQ ID NO: 1 - protein, 198 aa, Homo Sapiens
MSNVRVSNGSPSLERMDARQAEHPKPSACRNLFGPVDHEELTRDLEKHCRDMEEASQRKWNFDFQNHKPLEGKYEWQEVEKGSLPEFYYRPPRPPKGACKVPAQESQDVSGSRPAAPLIGAPANSEDTHLVDPKTDPSDSQTGLAEQCAGIRKRPATDDSSTQNKRANRTEENVSDGSPNAGSVEQTPKKPGLRRRQT

SEQ ID NO: 2 - DNA, 24 bp, ARTIFICIAL SEQUENCE
  PRIMER DIRECTED TO MOUSE SKP2
agagtggaagaacccaggcaggac

SEQ ID NO: 3 - DNA, 24 bp, ARTIFICIAL SEQUENCE
  PRIMER DIRECTED TO MOUSE SKP2
cccgtggagggaaaaagagggacg

SEQ ID NO: 4 - DNA, 24 bp, ARTIFICIAL SEQUENCE
  PRIMER DIRECTED TO MOUSE SKP2
gcatcgccttctatcgccttcttg

SEQ ID NO: 5 - DNA, 24 bp, ARTIFICIAL SEQUENCE
  PRIMER DIRECTED TO MOUSE SKP2
ttcccacccccacatccagtcatt

SEQ ID NO: 6 - DNA, 21 bp, ARTIFICIAL SEQUENCE
  PRIMER DIRECTED TO MOUSE RB1
ctctagatcctctcattcttc

SEQ ID NO: 7 - DNA, 20 bp, ARTIFICIAL SEQUENCE
  PRIMER DIRECTED TO MOUSE RB1
ccttgaccatagcccagcac

SEQ ID NO: 8 - DNA, 19 bp, ARTIFICIAL SEQUENCE
  PRIMER DIRECTED TO MOUSE YFP
aagttcatctgcaccaccg

SEQ ID NO: 9 - DNA, 20 bp, ARTIFICIAL SEQUENCE
  PRIMER DIRECTED TO MOUSE YFP
tgctcaggtagtggttgtcg

SEQ ID NO: 10 - DNA, 30 bp, Artificial Sequence
  PRIMER DIRECTED TO MOUSE Y1
gagcaggtttgttggcagtcgtacacctcc

SEQ ID NO: 11 - DNA, 26 bp, Artificial Sequence
  PRIMER DIRECTED TO MOUSE Y1
ccaatatggcggtggaagggagctga

SEQ ID NO: 12 - DNA, 20 bp, Artificial Sequence
  PRIMER DIRECTED TO MOUSE SKP2
agctgctccttgggatcttt

SEQ ID NO: 13 - DNA, 20 bp, Artificial Sequence
  PRIMER DIRECTED TO MOUSE SKP2
acgtctgggtgcagattttt

SEQ ID NO: 14 - DNA, 18 bp, Artificial Sequence
  PRIMER DIRECTED TO MOUSE GADPH
ggatgatgttctgggcag

SEQ ID NO: 15 - DNA, 18 bp, Artificial Sequence
  PRIMER DIRECTED TO MOUSE GADPH
ggatgatgttctgggcag

(19) United States
(12) Patent Application Publication
(10) Pub. No.: US 2011/0178156 A1
(43) Pub. Date: Jul. 21, 2011

(54) Method of suppressing pRb deficiency-induced tumor formation

(76) Inventor: Liang Zhu, Ardsley, NY (US)

(21) Appl. No.: 12/930,861

(22) Filed: Jan. 19, 2011

Related U.S. Application Data
(60) Provisional application No. 61/336,298, filed on Jan. 20, 2010.

(51) Int. Cl. A61K 31/713; A61P 35/00; C12Q 1/48; G01N 33/53

(52) U.S. Cl. 514/44 A; 435/15; 435/7.1

(57) ABSTRACT

The present invention provides methods of determining a putative agent that inhibits tumorigenesis in retinoblastoma protein deficient cells, the methods comprising determining whether the putative agent decreases phosphorylation of threonine residue 187 of p27, decreases S-phase kinase-associated protein 2 interaction with p27 having a phosphorylated threonine residue 187, or an increase in apoptosis of retinoblastoma protein deficient cells. The present invention also provides the agent, the pharmaceutical composition, and methods of inhibiting, preventing and treating tumorigenesis in retinoblastoma deficient cells, the method comprising administration of the agent that decreases phosphorylation of threonine residue 187 of p27 or the S-phase kinase-associated protein 2 interaction with p27 having a phosphorylated threonine residue 187.

### CROSS-REFERENCE TO RELATED APPLICATIONS

This application claims benefit of U.S. Provisional Application No. 61/336,298, filed Jan. 20, 2010, the contents of which are hereby incorporated by reference.

### STATEMENT OF GOVERNMENT SUPPORT

This invention was made with government support under grant numbers RO1 CA87566, RO1 CA131421, ROI DK58640, and RO1 CA127901 awarded by the National Institutes of Health, U.S. Department of Health and Human Services. The government has certain rights in the invention.

### FIELD OF THE INVENTION

The present invention relates to methods of determining and using agents that suppress tumorigenesis.

### BACKGROUND OF THE INVENTION

Throughout this application various publications are referred to in parenthesis. Full citations for these references may be found at the end of the specification. The disclosures of these publications are hereby incorporated by reference in their entirety into the subject application to more fully describe the art to which the subject invention pertains.

Heterozygosity of the retinoblastoma gene Rb1 elicits tumorigenesis in susceptible tissues following spontaneous loss of the remaining functional allele. Inactivation of previously-studied pRb targets partially inhibited tumorigenesis in Rb1^{+/-} mice (1, 2, 3, 4, 5, 6).

The retinoblastoma protein pRb is a prototype tumor suppressor that is inactivated in most, if not all, human cancers. pRb suppresses tumorigenesis via repressing its targets. As such, tumorigenesis following pRb loss is the result of overly active pRb targets. Therefore, inactivation of pRb targets when pRb is lost should neutralize the effects of pRb loss and, consequently, prevent tumorigenesis. pRb targets are therefore logical intervention targets for the prevention and treatment of pRb deficient tumors. In previous studies, many pRb targets have been identified. Individual inactivation of some of these targets only delayed tumorigenesis following pRb loss in mouse models. However, inactivation of several pRb targets together showed lethality for mouse embryogenesis, indicating intolerable toxicity to normal physiology. Therefore, the determination of a pRb target whose inactivation counters pRb loss, preventing tumorigenesis, is desirable. However, the inactivation of the pRb target must not be lethal to cells without pRb loss. To date, no pRb target has been able to meet these requirements.

The present invention solves this problem by providing a pRb target whose inactivation completely prevents tumorigenesis following pRb loss via a synthetic lethality to the cells that incur pRb loss, but is inconsequential to cells containing intact pRb. These two properties indicate that this pRb target is ideal for intervention to prevent and treat pRb-deficient tumors.

### SUMMARY OF THE INVENTION

A method of treating a tumor in a subject comprising administering to the subject an amount of an agent effective to (1) inhibit S-phase kinase-associated protein 2 (“Skp2”) in the cells of the tumor in the subject, or (2) inhibit phosphorylation of threonine residue no. 187 of p27 in the cells of the tumor in the subject, so as to thereby treat the tumor in the subject.

A method of identifying an agent as an inhibitor of tumorigenesis in retinoblastoma protein (pRb)-deficient cells, the method comprising contacting a p27 with the agent and a kinase which phosphorylates p27, and quantifying the degree of phosphorylation of threonine residue 187 of p27 (“p27T187”) by the kinase, wherein a decrease in the degree in phosphorylation of the p27 as compared to a control indicates that the agent is an inhibitor of tumorigenesis in pRb-deficient cells, while a lack of decrease in phosphorylation of the p27 as compared to a control indicates that the agent does not inhibit tumorigenesis in pRb-deficient cells.

A method of identifying an agent as an inhibitor of tumorigenesis in retinoblastoma protein (pRb)-deficient cells, the method comprising contacting a p27 having a phosphorylated threonine residue 187 (p27T187p) with an S-phase kinase-associated protein 2 (Skp2) complex and the agent, and quantifying the interaction between the Skp2 complex and the p27T187p, wherein a decrease in Skp2 complex interaction with the p27T187p as compared to a control indicates that the agent is an inhibitor of tumorigenesis in pRb-deficient cells, while a lack of decrease in Skp2 complex interaction with the p27T187p as compared to a control indicates that the agent is not an inhibitor of tumorigenesis in pRb-deficient cells.

A method of identifying an agent as promoting apoptosis of retinoblastoma protein (pRb)-deficient tumor cells, the method comprising contacting a p27 with the agent and a kinase which phosphorylates p27, and quantifying the degree of phosphorylation of threonine residue 187 of p27 (p27T187) by the kinase,

wherein a decrease in the degree in phosphorylation of the p27 as compared to a control indicates that the agent is a promoter of apoptosis of retinoblastoma protein (pRb)-deficient tumor cells, while a lack of decrease in phosphorylation of the p27 as compared to a control indicates that the agent is not a promoter of apoptosis of retinoblastoma protein (pRb)-deficient tumor cells.

A method of identifying an agent as promoting apoptosis of retinoblastoma protein (pRb)-deficient tumor cells, the method comprising contacting a p27 having a phosphorylated threonine residue 187 (p27T 187p) with an S-phase kinase-associated protein 2 (Skp2) complex and the agent, and quantifying the interaction between the Skp2 complex and the p27T187p,

wherein a decrease in Skp2 complex interaction with the p27T187p as compared to a control indicates that the agent is a promoter of apoptosis of retinoblastoma protein (pRb)-deficient tumor cells, while a lack of decrease in Skp2 complex interaction with the p27T187p as compared to a control indicates that the agent is not a promoter of apoptosis of retinoblastoma protein (pRb)-deficient tumor cells.

### BRIEF DESCRIPTION OF THE DRAWINGS

FIG. 1A-1E. Roles of Skp2 in spontaneous tumorigenesis in Rb1^{+/-} mice and in ENU-induced tumorigenesis. (A) Expression of the indicated proteins in wild type normal pituitary glands and pituitary tumors developed in Rb1^{+/-}Skp2^{+/+} mice, determined by Western blot. (B) Levels of Skp2 mRNA in pituitary glands and pituitary tumors (developed in Rb1^{+/-} mice), determined by Q-PCR normalized with GAPDH. (C) Incidence for pituitary and thyroid tumors at various stages in Rb1^{+/-}Skp2^{+/+} and Rb1^{+/-}Skp2^{-/-} mice. p values are by Fisher's exact tests (various lesions were combined for analyses). (D) Kaplan-Meier survival analysis for the indicated mice. p value is by Log Rank test. One Rb1^{+/-}Skp2^{-/-} mouse died at thirteen months and one died at sixteen months with macroscopically normal pituitary and thyroid glands. The causes of death were unclear with a possible association with eye and skin lesions. (E) Kaplan-Meier survival analysis for the indicated mice treated with ENU.

FIG. 2A-2B. Effects of Skp2 inactivation on E2F deregulation, aberrant proliferation and apoptosis, and p27 expression in pituitary ILs following Rb1 deletion. Quantification of Ki67 (A) and TUNEL (B) labeling in ILs was performed with three pituitaries of each indicated genotypes at the indicated ages. Rb1 genotypes indicate the outcome of Cre-loxP mediated deletion in IL. p values are by t test. Error bars are s.d.

FIG. 3A-3B. Effects of targeted deletion of Rb1 in pituitary IL of p27T187A KI mice. Quantification of Ki67 (A) and TUNEL (B) labeling in ILs following Rb1 deletion at the indicated ages. p values are by t test. Error bars are s.d.

FIG. 4A-4L. Effects of Skp2 knockdown and stabilized p27 expression on established Y79 cells and early passage RB177 retinoblastoma cells. (A-E) Y79 and RB177 cells infected with lentiviruses expressing shRNA targeting Skp2. Two independent Skp2 shRNAs and a scrambled shRNA control (Scrm) were used as indicated. After drug selection, infected cells were evaluated for Skp2 mRNA by quantitative RT-PCR (A), cell proliferation by counting live cells (B), cell cycle profile by FACS (C), apoptosis by TUNEL staining (D), and p27 expression by Western immunoblotting, with Cdk2 as a loading control (E). (F-I) Y79 and RB177 cells infected with BE-GFP lentiviral vector encoding p27 or p27T187A. Infected cells were evaluated for p27 expression (F), cell proliferation (G), cell cycle profile (H), and TUNEL staining (I). (J-K) Y79 cells transduced with BE-GFP vector or BE-GFP-RB, followed 2 days later by transduction with Skp2 shRNA or scrambled shRNA control (J) or with BE-GFP or BE-GFP-p27T187A (K), and evaluated cells with sub-G1 DNA content. Averages with s.d. are shown. Asterisks indicate P<0.05 relative to applicable controls. (L) A new model of tumorigenesis after Rb1 loss. Two consecutive arrows suggest the presence of multiple steps between them.

FIG. 5A-5C. Effects of Skp2 knockout in the pituitary gland intermediate lobe in comparison to the liver. Unlike the livers in the same animals pituitary glands of 6-month old Skp2^{-/-} mice did not contain enlarged nuclei or detectably higher levels of p27 protein in melanotrophs (C). Anti-p27 Western blotting with extracts of liver and pituitary glands of Skp2^{+/+} and Skp2^{-/-} mice confirmed results obtained by anti-p27 IHC (C). To directly determine the effects of Skp2 knockout on cell proliferation in pituitary glands, the BrdU incorporation rates in pituitaries of Skp2^{+/+} and Skp2^{-/-} embryos were measured at 18.5 days of gestation and found them to be similar (B). Thus, Skp2 is not required for normal organogenesis and homeostasis of the pituitary gland including its intermediate lobe. Anterior lobe (AL), intermediate lobe (IL), and posterior lobe (PL). Quantification of nuclear sizes (areas) of about 600 cells from three Skp2^{+/+} or Skp2^{-/-} mice was by Axiovision (Zeiss) image analysis software. “Hepato”, hepatocytes; “Melano”, melanotrophs of IL. Averages and s.d. are shown. Quantification of BrdU labeling was performed with three embryos each of the indicated genotypes.

FIG. 6A. Skp2 inactivation does not protect mice from ENU induced tumorigenesis. Protein expression levels for Skp2 and p27 in representative normal spleen and lymphoma tissues were determined by Western Blot.

FIG. 7A-7D. Effects of p14Arf knockdown and Skp2 knockdown on early passage RB177 retinoblastoma cells. (A) RB177 cells were infected with lentiviruses expressing shRNA targeting p14Arf or scrambled shRNA (Scrm), and p14Arf mRNA levels determined by Q-RT-PCR. (B) Cells from (A) were infected with lentiviruses expressing shRNA targeting Skp2 or Scrm shRNA, and Skp2 mRNA levels determined by Q-RT-PCR on day 4. (C) Cell numbers measured on the indicated days for cells treated as indicated. (D) Cell cycle profile by FACS, including sub-G1 cell fraction for apoptotic cells. Skp2 knockdown-induced increase in sub-G1 fraction was not diminished by co-knockdown of p14Arf.

### DETAILED DESCRIPTION OF THE INVENTION

A method of treating a tumor in a subject comprising administering to the subject an amount of an agent effective to (1) inhibit S-phase kinase-associated protein 2 (“Skp2”) in the cells of the tumor in the subject, or (2) inhibit phosphorylation of threonine residue no. 187 of p27 in the cells of the tumor in the subject, so as to thereby treat the tumor in the subject.

In an embodiment, the subject is administered an agent which inhibits Skp2 in the cells of the tumor in the subject. In an embodiment, the agent is a double-stranded siRNA molecule directed against a nucleic acid encoding Skp2. In an embodiment, the subject is administered an agent which inhibits phosphorylation of threonine residue no. 187 of p27 in the cells of the tumor. In an embodiment, the tumor is a tumor comprising retinoblastoma protein (pRb)-deficient cells. In an embodiment, the tumor is a tumor comprising Rb1 +/− cells and/or Rb1 −/− cells. In an embodiment, the tumor is a tumor of the subject's retina, bone, lung, prostate, breast, bladder, brain, oesophagus, or liver.

A method of identifying an agent as an inhibitor of tumorigenesis in retinoblastoma protein (pRb)-deficient cells, the method comprising contacting a p27 with the agent and a kinase which phosphorylates p27, and quantifying the degree of phosphorylation of threonine residue 187 of p27 (“p27T187”) by the kinase,

wherein a decrease in the degree in phosphorylation of the p27 as compared to a control indicates that the agent is an inhibitor of tumorigenesis in pRb-deficient cells, while a lack of decrease in phosphorylation of the p27 as compared to a control indicates that the agent does not inhibit tumorigenesis in pRb-deficient cells.

A method of identifying an agent as an inhibitor of tumorigenesis in retinoblastoma protein (pRb)-deficient cells, the method comprising contacting a p27 having a phosphorylated threonine residue 187 (p27T187p) with an S-phase kinase-associated protein 2 (Skp2) complex and the agent, and quantifying the interaction between the Skp2 complex and the p27T187p,

wherein a decrease in Skp2 complex interaction with the p27T 187p as compared to a control indicates that the agent is an inhibitor of tumorigenesis in pRb-deficient cells, while a lack of decrease in Skp2 complex interaction with the p27T187p as compared to a control indicates that the agent is not an inhibitor of tumorigenesis in pRb-deficient cells.

A method of identifying an agent as promoting apoptosis of retinoblastoma protein (pRb)-deficient tumor cells, the method comprising contacting a p27 with the agent and a kinase which phosphorylates p2′7, and quantifying the degree of phosphorylation of threonine residue 187 of p27 (p27T187) by the kinase, wherein a decrease in the degree in phosphorylation of the p27 as compared to a control indicates that the agent is a promoter of apoptosis of retinoblastoma protein (pRb)-deficient tumor cells, while a lack of decrease in phosphorylation of the p27 as compared to a control indicates that the agent is not a promoter of apoptosis of retinoblastoma protein (pRb)-deficient tumor cells.

A method of identifying an agent as promoting apoptosis of retinoblastoma protein (pRb)-deficient tumor cells, the method comprising contacting a p27 having a phosphorylated threonine residue 187 (p27T187p) with an S-phase kinase-associated protein 2 (Skp2) complex and the agent, and quantifying the interaction between the Skp2 complex and the p27T187p,

wherein a decrease in Skp2 complex interaction with the p27T187p as compared to a control indicates that the agent is a promoter of apoptosis of retinoblastoma protein (pRb)-deficient tumor cells, while a lack of decrease in Skp2 complex interaction with the p27T187p as compared to a control indicates that the agent is not a promoter of apoptosis of retinoblastoma protein (pRb)-deficient tumor cells.

In an embodiment of the methods, the kinase is a cyclin-dependent kinase (Cdk). In an embodiment of the methods, the degree of phosphorylation of p27T187 or the degree of Skp2 complex interaction with p27T187p is measured by a binding assay. In an embodiment of the methods, the binding assay is a fluorescence-based binding assay or an immunospecific binding assay. In an embodiment of the methods, the p27 is p27Kip1.

In an embodiment of the methods described herein, the subject is a human.

pRb is a tumor suppressor protein found in all human cell lines. It is inactivated in most, if not all, human cancers. pRb-deficient cells may spontaneously arise from heterozygous Rb1^{+/-} cells. pRb-deficient cells, found in many tumors, are cells in which pRb expression or pRb levels are below that of normal cells, or absent.

pRb-deficient cells may either be cells grown in vivo or in vitro which spontaneously inactivate pRb. Tissues most susceptible to pRb-loss include retina, bone, lung, prostate, breast, bladder, esophagus, and liver. Alternatively, the cells, in vivo or in vitro may be manipulated by any physical or chemical method known in the art to inactivate pRb. For example, the pRb gene of the cells may be mutated by any method known in the art, such as by deleting the entire pRb gene, or deleting or substituting portions of the pRb gene. The mutation may be either heterozygous or homozygous. Mutations which are of use in the present invention are those in which the pRb protein is absent or inactivated.

p27 is a protein which is a target of pRb. Specifically, p27 as used herein refers to p27^{Kip1}, i.e. p27 kinase inhibitor protein 1. Decreased p27 protein levels are found to correlate with more aggressive tumor progression and a worse prognosis. Cyclin-dependent kinase (Cdk) is a protein kinase which phosphorylates proteins on serine and threonine residues. Skp2 (S-phase kinase-associated protein 2) is an enzyme which is part of a ubiquitin ligase complex (Skp2 complex) which includes cyclin-dependant kinases regulatory protein 1 (Cks1), S-phase kinase-associated protein 1 (Skp1), and Cul1. When threonine (T) residue 187 (T187) of p27 is phosphorylated (p27T187p), the Skp2 complex interacts with the p27 by ubiquitinating the p27 protein. This interaction between Skp2 and p27T187p marks the p27 protein for degradation by the cell. A high level of p27 in cells which are pRb-deficient causes apoptosis. Inhibiting phosphorylation of p27T187 is lethal to pRb-deficient cells with little to no effect on non-pRb-deficient cells. As used herein, “p27T187p” is phosphorylated p27T187.

The sequence for human p27^{Kip1} is as follows: MSNVRVSNGS PSLERMDARQ AEHPKPSACR NLFGPVDHEE LTRDLEKHCR DMEEASQRKW NFDFQNHKPL EGKYEWQEVE KGSLPEFYYR PPRPPKGACK VPAQESQDVS GSRPAAPLIG APANSEDTHL VDPKTDPSDS QTGLAEQCAG IRKRPATDDS STQNKRANRT EENVSDGSPN AGSVEQTPKK PGLRRRQT (SEQ ID NO:1) The sequence is available at the National Center for Biotechnology Information (NCBI) website. The threonine 187 residue of p27^{Kip1} is conserved in humans, pigs, mice, chickens, hamsters, and xenopus.

The term “agent” or “putative agent” as used herein, unless otherwise indicated by context, means a chemical or biological agent. In non-limiting examples, an agent or putative agent is a chemical, small molecule (i.e organic and 800 daltons or less in mass), a polypeptide, a protein, a protein fragment, a peptide mimetic, a monoclonal antibody or an antigen-binding fragment thereof, an siRNA or shRNA, or an aptamer. Preferably, the agent is biomembrane-permeable. An aptamer may be a single stranded oligonucleotide or oligonucleotide analog that binds to a particular target molecule, such as a protein. Alternatively, an aptamer may be a protein aptamer which consists of a variable peptide loop attached at both ends to a protein scaffold that interferes with protein interaction. A peptide mimetic is a short peptide which mimics the sequence of a protein of interest. Preferably, the peptide mimetic is a short peptide which mimics the sequence of p27 and which is preferably centered on threonine residue 187. Such a peptide mimetic interacts with Cdk and the Skp2 complex in the same manner as p27. The interaction of the peptide mimetic with Cdk and the Skp2 complex may result in the inhibition of the phosphorylation of p27T187 and/or the inhibition of the interaction between Skp2 and p27T187p, for example, by competition. Preferably, the agent inhibits the phosphorylation of p27T187 or inhibits the interaction between Skp2 and p27T187p without inhibiting, or only nominally inhibiting, the phosphorylation or ubiquination of other kinase substrates.

p27 and Cdk may be combined with the putative agent and the degree of phosphorylation of the threonine 187 residue of p27 may be determined. Similarly, p27T187p and Skp2 complex may be combined with the putative agent and the degree of Skp2 interaction with p27T187p may be determine, for example, by measuring the degree of ubiquitination of the p27T187p. A decrease in the degree of phosphorylation of p27T187 or a decrease in Skp2 interaction with p27T187p indicates that the putative agent inhibits tumorigenesis of pRb-deficient cells. A lack of decrease in the degree of phosphorylation of p27T187 or lack of decrease in Skp2 interaction with p27T187p indicates that the putative agent does not inhibit tumorigenesis of pRb-deficient cells.

An inhibitor of Skp2 includes, in non-limiting examples, small molecules (i.e. organic molecules with a molecular mass of 800 daltons or less), monoclonal antibodies, an antigen-binding fragments thereof, directed against Skp2, and RNAi such as shRNA and siRNA which inhibit expression or function of Skp2. shRNA targeting Skp2 are, for example, set forth in Lin et al., (2010). Inhibitors of phosphorylation of p27T187 include small molecules (i.e. organic molecules with a molecular mass of 800 daltons or less), monoclonal antibodies directed against T187 of p27, and RNAi such as shRNA and siRNA.

The degree of phosphorylation of p27 (at residue 187) or the degree of Skp2 complex interaction with p27T187p can be measured by any method known in the art including, but not limited to, by assay. Any assay known in the art may be used such as, for example, a binding assay. Examples of binding assays include, but are not limited to, fluorescence-based assays and immunospecific assays. When whole cells or tumors have been contacted with the putative agent, the assay may be performed on the whole cell or the p27, p27T187p, and ubiqutinated p27T187p may be freed from the cells by any method known in the art including, but not limited to, cell lysing. Additionally, the p27, p27T187p, and ubiquitinated p27T187p may be purified before performing the assay.

The degree of phosphorylation of p27 (at residue 187) or of Skp2 complex interaction with p27T187p of the p27 and Cdk or p27T187p and Skp2 complex contacted with the putative agent may be compared to the degree of phosphorylation or Skp2 interaction with p27T187p of one or more controls.

A control as used herein means a reference standard which is predetermined or which is generated or quantified under conditions identical to that which it is being compared to, except that the agent being investigated is absent. For example, when determining if an agent inhibits phosphorylation of p27T187, contacting p27 with a kinase in the absence of the agent, and measuring the degree of phosphorylation of p27T187 is a control for comparing the phosphorylation of p27T187 under identical or very similar conditions (i.e. contacting p27 with the kinase and other conditions being the same) but with the presence of the agent.

The cells as referred to in the present invention may be located in vivo or in vitro. The cells may be separate cells or may comprise, or be part of, a tumor. If in vivo, the cells or tumor of the present invention may be in a human. In another embodiment, the in vivo cells are a xenograft of human cells in a mammalian model. Preferably, the mammalian model is a rodent. In the case of a xenograft, the cells may be contacted with the putative agent directly after graft of the cells, or a tumor may be allowed to form before contacting the cells with the putative agent.

The pRb-deficient cells may be contacted by the putative agent by any method known in the art. For example, when the pRb-deficient cells are located in vivo, the method of contacting may include, but is not limited to, infusing the putative agent into the circulatory system or injection of the putative agent directly into, or near, the tumor.

There are many ways known in the art to measure cell activity. For example, cell activity may be measured by apoptosis, or lack thereof. Apoptosis of the pRb-deficient cells contacted with the putative agent indicates that the putative agent inhibits the phosphorylation of p27T187 or the interaction between Skp2 and p27T187p. Continued cell growth, or lack of apoptosis, indicates that the putative agent does not inhibit the phosphorylation of p27T187 or the interaction between Skp2 and p27T187p. Apoptosis may be measured by any method known in the art, for example, TUNEL (terminal d-UTP nicked-end labeling) assay, immunohistochemistry staining for apoptotically-expressed proteins, “comet-tail” assay, measuring tumor thinning, or counting pyknotic nuclei. Other methods of measuring cell activity include, but are not limited to, measuring cell growth, cell division, or uptake of nutrients. Cell activity can be measured by any method known in the art and the method used will depend on the type of cell activity being measured. For example, when the cells being contacted with the putative agent are in vitro, cell activity may be measured by counting live cells, microscopy or flouroscopy. Alternatively, when the cells being contacted with the putative agent are an in vivo tumor, apoptosis may be measured by, for example, taking a tissue sample of the tumor and measuring thinning of the tumor before and after contacting the tumor with the putative agent, microscopy, or flouroscopy. When there is a control, cell activity of the pRb-deficient cells contacted with the putative agent can be compared with the cell activity of the control. The control comprises pRb-deficient cells which are not contacted with the putative agent.

The present invention may be performed with high throughput arrays, such as a 384-well plate format.

The present invention provides an agent for inhibiting tumorigenesis in retinoblastoma protein (pRb) deficient cells, the agent determined by: (1) contacting p27 with Cdk and the putative agent and measuring the degree of phosphorylation; (2) contacting p27T187p with Skp2 complex and the putative agent and measuring Skp2 interaction with p27T187p; (3) contacting pRb-deficient cells with the putative agent and measuring cell activity; or (4) contacting pRb deficient cells with the putative agent and measuring the degree of phosphorylation or Skp2 interaction with p27T187p, wherein a decrease in the degree of phosphorylation or Skp2 interaction with p27T187p or a change in cell activity indicates that the putative agent inhibits tumorigenesis in pRb-deficient cells, while a lack of decrease in the degree of phosphorylation or Skp2 interaction with p27T187p or a lack of change in cell activity indicates that the putative agent does not inhibit tumorigenesis in pRb-deficient cells.

The cells may be in vivo or in vitro and may comprise a tumor. Measuring cell activity may comprise measuring apoptosis which may be measured by counting cells or measuring thinning of the tumor. Cell activity of a control, comprising pRb-deficient cells which are not contacted with the putative agent, may be compared to the cell activity of the pRb-deficient cells contacted with the putative agent.

The agent may be associated with a pharmaceutically-acceptable carrier, thereby comprising a pharmaceutical composition. The pharmaceutical composition may comprise the agent in a pharmaceutically acceptable carrier. Alternatively, the pharmaceutical composition may consist essentially of the agent in a pharmaceutically acceptable carrier. Yet alternatively, the pharmaceutical composition may consist of the agent in a pharmaceutically acceptable carrier.

The pharmaceutically-acceptable carrier must be compatible with the agent, and not deleterious to the subject. Examples of acceptable pharmaceutical carriers include carboxymethylcellulose, crystalline cellulose, glycerin, gum arabic, lactose, magnesium stearate, methyl cellulose, powders, saline, sodium alginate, sucrose, starch, talc, and water, among others. Formulations of the pharmaceutical composition may conveniently be presented in unit dosage and may be prepared by any method known in the pharmaceutical art. For example, the agent may be brought into association with a carrier or diluent, as a suspension or solution. Optionally, one or more accessory ingredients, such as buffers, flavoring agents, surface active ingredients, and the like, may also be added. The choice of carriers will depend on the method of administration. The pharmaceutical composition would be useful for administering the agent to a subject to prevent or treat tumorigenesis. The agent is provided in amounts effective to prevent or treat tumorigenesis in the subject. These amounts may be readily determined by one in the art. In one embodiment, the agent is the sole active pharmaceutical ingredient in the formulation or composition. In another embodiment, there may be a number of active pharmaceutical ingredients in the formulation or composition aside from the putative agent. In this embodiment, the other active pharmaceutical ingredients in the formulation or composition must be compatible with the agent.

The present invention also provides a method of inhibiting tumorigenesis in retinoblastoma protein (pRb) deficient cells, the method comprising contacting pRb-deficient cells with an agent that decreases the degree of phosphorylation of threonine residue 187 of p27 (p27T187) or S-phase kinase-associated protein 2 (Skp2) interaction with p27 having a phosphorylated threonine residue 187 (p27T187p).

The present invention further provides a method of preventing or treating tumorigenesis in retinoblastoma protein (pRb) deficient cells, the method comprising administering to a subject with pRb-deficient cells a therapeutically effective amount of an agent that decreases the degree of phosphorylation of threonine residue 187 of p27 (p27T187) or S-phase kinase-associated protein 2 (Skp2) interaction with p27 having a phosphorylated threonine residue 187 (p27T187p).

The pRb-deficient cells may be in vitro or in vivo and may have formed a tumor. In the present invention, the agent is administered to the pRb-deficient cells in an amount and manner which is effective to prevent or treat tumorigenesis in the pRb-deficient cells. Effective to prevent means effective to prevent pRb-deficient cells from forming a tumor. Effective to treat tumorigeneisis or treat a tumor means effective to reduce, minimize or reverse the clinical pathology of a pRb-deficient tumor or cell mass. The amount of agent effective to prevent or treat tumorigenesis will vary depending on the presence of pRb-deficient cell masses or tumors, the clinical pathology of any such pRb-deficient cell masses or tumors, and the type of putative agent. Appropriate amounts of the agent effective to prevent or treat tumorigenesis can be readily determined by the skilled artisan without undue experimentation. Additionally, the manner of administration of the agent which is effective to prevent or treat tumorigenesis will depend on the location of the pRb-deficient cell mass or tumor, if any. The manner of administration must allow the agent to reach the pRb-deficient cells, cell mass, or tumor. Since pRb is present in all human cell lines, pRb-deficient cells, cell mass, or tumor may be found anywhere in the human body. For example, when the pRb-deficient cells, cell mass, or tumor is in the brain, the agent must be administered in a manner that either allows the agent to cross the blood-brain barrier or must be administered directly into the brain, or by nasal administration. Alternatively, the pRb-deficient cells, cell mass, or tumor may be located within the torso, internal organs, appendages, or other locations. The agent can be administered in any manner that would allow the agent to reach the pRb-deficient cells, cell mass, or tumor, such as by injection.

The agent is to be administered in an amount and manner effective to prevent or treat tumorigenesis in pRb-deficient cells. According to the methods of the present invention, the agent may be administered to a subject by any method known in the art including, but not limited to, parenteral administration. For a parenteral administration, the agent may be combined with sterile aqueous solution which is preferably isotonic with the blood of the subject. Such a formulation may be prepared by dissolving a solid active ingredient in water containing physiologically-compatible substances, such as sodium chloride, glycine, and the like, and having a buffered pH compatible with physiological conditions, so as to produce an aqueous solution, then rendering said solution sterile. The formulations may be delivered by any mode of injection including, without limitation, epifascial, intrasternal, intravascular, intravenous, parenchymatous, or subcutaneous.

The present invention provides the use of a pharmaceutical that inhibits the phosphorylation of p27T187 for the treatment or prevention of tumorigenesis in pRb-deficient cells. The present invention also provides the use of a pharmaceutical that inhibits the interaction between Skp2 and p27T187p for the treatment or prevention of tumorigenesis in pRb-deficient cells. The present invention further provides the use of an inhibitor of the phosphorylation of p27T187 for the preparation of a medicament for the treatment or prevention of tumorigenesis in pRb-deficient cells. The present invention additionally provides the use of an inhibitor of the interaction between Skp2 and p27T187p for the preparation of a medicament for the treatment or prevention of tumorigenesis in pRb-deficient cells.

### Experimental Details

### 1. Methods and Materials

Mice. Rb1^{+/-} mice and Skp2^{+/-} mice have been previously described (12, 14). Mouse strain background is as follows. Skp2^{+/-} mice on mixed C57BL/6Jx129Sv strain background were backcrossed to C57BL/6J strain mice four times, and Rb1^{+/-} mice on mixed C57BL/6Jx129Sv strain background were backcrossed to C57BL/6J mice once. Rb1^{+/-}Skp2^{+/-} mice were then generated from these mice and were used to generate littermate Rb1^{+/-}Skp2^{+/+} and Rb1^{+/-}Skp2^{-/-} mice. These Rb1+/− mice may therefore exhibit a slower tumor development kinetics than Rb1^{+/-} mice with equal contributions from C57BL/6J and 129Sv strain background (20). Rb1 heterozygous mice were genotyped according to a published protocol (12). POMC-Cre transgenic mice were genotyped as previously described (21). Primers for genotyping Skp2^{+/-} mice, Rb1 lox/lox mice (22), Rosa26R(YFP) mice (23), and p27T187A KI mice (15) are listed in Table 1.

TABLE 1
Oligonucleotide Primers
Skp2 KO mice genotyping

| | |
|---|---|
| Skp2WT-5′ | 5-AGAGTGGAAGAACCCAGGCAGGAC-3 |
| | (SEQ ID NO: 2) |
| Skp2WT-3′ | 5-CCCGTGGAGGGAAAAAGAGGGACG-3 |
| | (SEQ ID NO: 3) |
| Skp2MUT-5′ | 5-GCATCGCCTTCTATCGCCTTCTTG-3 |
| | (SEQ ID NO: 4) |
| Skp2MUT-3′ | 5-TTCCCACCCCCACATCCAGTCATT-3 |
| | (SEQ ID NO: 5) |

Rb1 lox/lox mice genotyping

| | |
|---|---|
| Rb5lox | 5-CTCTAGATCCTCTCATTCTTC-3 |
| | (SEQ ID NO: 6) |
| Rb3lox | 5-CCTTGACCATAGCCCAGCAC-3 |
| | (SEQ ID NO: 7) |

Rosa26R(YFP) mice genotyping

| | |
|---|---|
| YFP1 | 5-AAGTTCATCTGCACCACCG-3 |
| | (SEQ ID NO: 8) |
| YFP2 | 5-TGCTCAGGTAGTGGTTGTCG-3 |
| | (SEQ ID NO: 9) |

p27T187A KI mice genotyping

| | |
|---|---|
| Y1 | 5-GAGCAGGTTTGTTGGCAGTCGTACACCTCC-3 |
| | (SEQ ID NO: 10) |
| H3 | 5-CCAATATGGCGGTGGAAGGGAGCTGA-3 |
| | (SEQ ID NO: 11) |

mSkp2 Q-PCR

5-AGCTGCTCCTTGGGATCTTT-3
(SEQ ID NO: 12)
5-ACGTCTGGGTGCAGATTTTT-3
(SEQ ID NO: 13)

mGAPDH Q-PCR

5-GGATGATGTTCTGGGCAG-3
(SEQ ID NO: 14)
5-GGATGATGTTCTGGGCAG-3
(SEQ ID NO: 15)

The animals studied for ENU mutagenesis were C57BL/6Jx129Sv hybrid strain littermate mice from Skp2 heterozygous crosses. Skp2^{+/+} and Skp2^{-/-} mice were i.p. injected with ENU (0.5 mmol per gram of body weight) at PND 15±2 days as previously described (24). Mice were sacrificed at the first sign of morbidity, which included abdominal swelling, hunched posture, and rapid breathing. Complete necropsies of all internal organs were performed including size measurement of tumors.

All mouse study protocols were approved by the Albert Einstein College of Medicine Animal Institute.

Western blot and RT-PCR analyses. Normal pituitaries, fractions of gross pituitary tumors, and fractions of ENU-induced tumors were snap frozen in ethanol-dry ice and stored in −80° C. For Western blot, frozen tissues were homogenized with Dounce glass homogenizer in tissue lysis buffer (50 mM HEPES pH7.2, 150 mM NaCl, 1 mM EDTA, 0.1% Tween®-20, 1 mM DTT, and standard protease inhibitors). Tissue debris was removed by centrifugation for 10 minutes at 14,000 rpm at 4° C. Protein concentrations of the extracts were determined by Bio-Rad protein assay kit and equal amounts of protein samples were loaded on 10% SDS gels, blotted onto PVDF membrane. Antibodies to Skp2 (H435), p27 (C-19), cyclin A (C-19), cyclin E (M-20), and Cdk2 (C-19) were from Santa Cruz Biotechnology (Santa Cruz, Calif.).

For Q-PCR, tissue RNA was extracted by TRIzol® reagent (Invitrogen, now Life Technologies, Carlsbad, Calif.). Total RNA was treated with RQ1 DNase (Promega, Madison, Wis.) at 37° C. for 30 min. and RQ1 was denatured at 65° C. for 20 min. T7 oligonucleotides and SuperScript™ II (Invitrogen now Life Technologies, Carlsbad, Calif.) were used for the synthesis of the first strand cDNA at 42° C. for 60 minutes. The PCR primers for mSkp2 and mGAPDH are listed in Table 1. SYBR® Green PCR Master Mix (4309155, ABI) and the standard program of ABI Prism® 7000 were used for Q-PCR amplification.

Immunohistochemistry staining and frozen sectioning for fluorescence detection. Paraffin sections were stained with Histomouse™-plus kit (ZYMED®) with antibodies to PCNA (PC10) and p27 (C-19) from Santa Cruz Biotechnology (Santa Cruz, Calif.), and to BrdU (Ab-2) from Calbiochem (EMD, Gibbsotwn, N.J.) and Ki67 as primary antibody (1 μg/ml). TUNEL staining was performed with the reagents and instructions of Apoptosis Detection Kit (S7101) from Chemicon (now Millipore, Billerica, Mass.).

Pituitaries were fixed in 4% paraformaldehyde, 10% glucose in PBS for 30 minutes and embed in Tissue Freezing Medium (H-TFM, Triangle Biomedical Sciences, Durham, N.C.) on dry ice for frozen sectioning. After fluorescence photography, slides were counter-stained by Hematoxylin.

Lentivirus infection and analysis of human retinoblastoma cells. Y79 cells were purchased from the ATCC and RB177 cells were derived from a human retinoblastoma and passaged for approximately 2 months, with no evidence of a crisis phase, prior to the knockdown analyses (19). Skp2 shRNAs were delivered by pLKO constructs TRCN0000007530 and TRCN0000007534 (Open Biosystems, Huntsville, Ala.), and were compared to a pLKO encoding a non-silencing control shRNA (Addgene, Cambridge, Mass.). RB177 cells with constitutive CDKN2A^{ARF} knockdown and pLKO-transduced controls were as described (19). pRb, p27, and p27T187A were delivered using the bidirectional BE-GFP vector (25). BE-GFP-p27+3′ and BE-GFP-p27T187A+3′ were produced by inserting a XmaI-XbaI fragment of pCS+p27 and pCS+p27(T187A) (26) extending from the p27 coding region to the 3′ UTR between the corresponding XmaI site and a vector XbaI site of BE-GFP-p27 (25). BE-GFP-Rb was as described (25). Cells were cultured, infected, and analyzed as described (19).

Statistics analysis. In the survival analysis, difference in Kaplan-Meier survival curves was analyzed by Log Rank Test (JMP software, Cary, N.C.). Differences in gross tumor incidence, incidence of microscopic lesions in macroscopically normal pituitary and thyroid glands were analyzed by Fisher's exact test (MedCalc software, Mariakerke, Belgium). Differences in TUNEL labeling indices between Rb1^{lox/lox};POMC-Cre;Skp2^{+/+} and Rb1^{lox/lox};POMC-Cre;Skp2^{-/-} ILs and between Rb1^{lox/lox};POMC-Cre;p27^{+/+} and Rb1^{lox/lox};POMC-Cre;p27^{T187A/T187A} ILs were analyzed by student's t-test (MedCalc Software, Mariakerke, Belgium).

### 2. Results

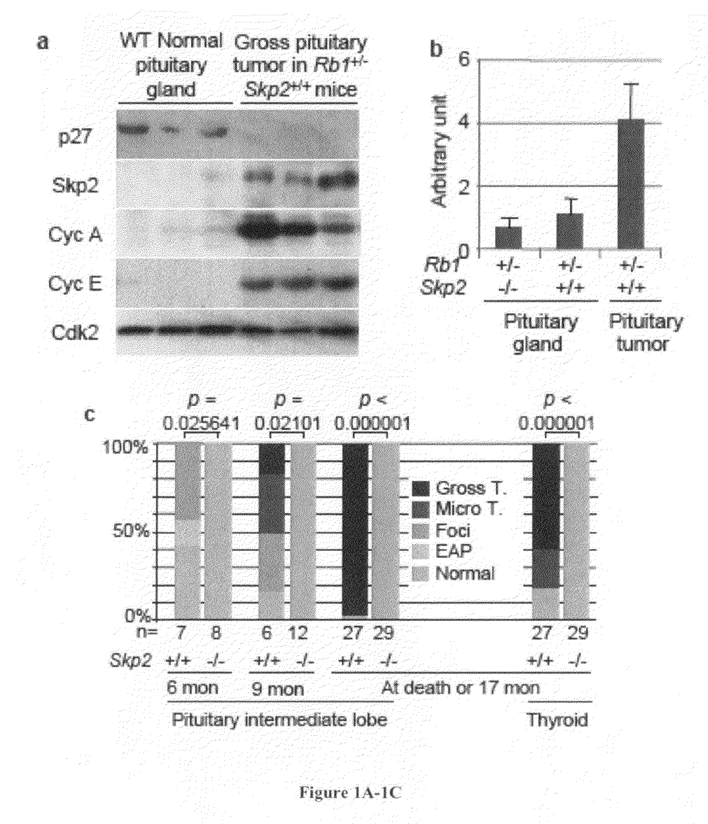

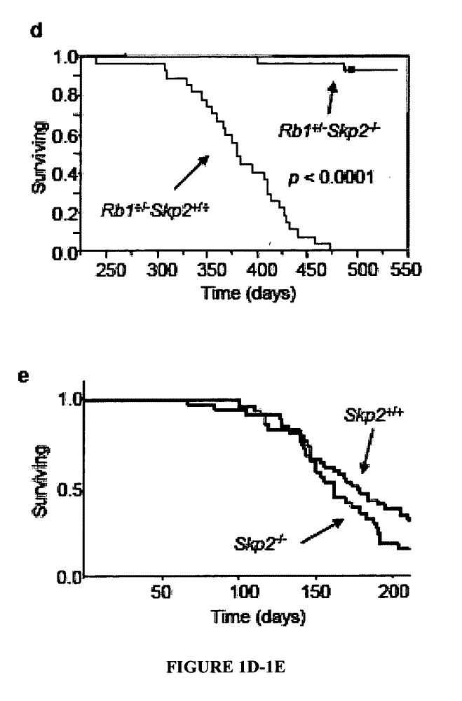

To define the role of Skp2 in tumorigenesis in Rb1^{+/-} mice, cohorts of Rb1^{+/-} Skp2^{+/+} and Rb1^{+/-}Skp2^{-/-} mice were generated. Skp2 is not required for pituitary gland development. Rb1^{+/-} mice develop pituitary intermediate lobe (IL) melanotroph tumors with a well-defined course, from early atypical proliferates (EAP) to foci, microscopic tumors, and gross tumors, resulting in death around one year of age (12). At 6-month, more than half of Rb1^{+/-}Skp2^{+/+} mice had EAPs and foci (FIG. 1C). By 9 months, one pituitary had a gross tumor, while most had foci and microscopic tumors. Later, all 27 Rb1^{+/-}Skp2^{+/+} mice died between 10 to 15 months of age (FIG. 1D), with gross pituitary tumors except for one (FIG. 1C). In contrast, none of the Rb1^{+/-}Skp2^{-/-} mice had any sign of pituitary tumorigenesis at 6, 9, and 17 months, when healthy Rb1^{+/-}Skp2^{-/-} mice were sacrificed.

Thyroid C-cell tumors develop with 50-70% penetrance in Rb1^{+/-} mice. Sixteen of the same 27 Rb1^{+/-}Skp2^{+/+} mice had gross thyroid tumors at death, and the dead mouse that lacked a pituitary tumor had an especially large thyroid tumor (FIG. 1C). About half of the remaining dead mice had microscopic thyroid tumors (FIG. 1C). In contrast, all 29 Rb1^{+/-}Skp2^{-/-} mice had normal appearing tumor-free thyroid glands (FIG. 1C). Together with the lack of pituitary tumors, these results identify Skp2 as the first pRb target that is required for spontaneous tumorigenesis in Rb1^{+/-} mice.

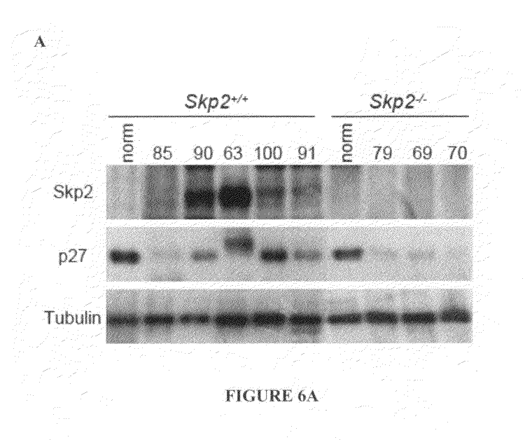

The above findings could reflect that Skp2 plays a required role in the development of Rb1 mutant tumors or that Skp2 is generally required for tumorigenesis. To investigate, Skp2^{+/+} and Skp2^{-/-} mice were treated with an ENU-induced tumorigenesis protocol. No difference in tumor development in the two genotypes was demonstrated, including survival (FIG. 1E) and tumor types and burdens (Table 2). Although Skp2 was frequently overexpressed in the tumors, its expression levels did not correlate with p27 protein levels (FIG. 6A). Thus, Skp2 is not required for ENU-induced tumorigenesis.

TABLE 2

| | Sub-mandible Lymphoma | | | Thoracic Lymphoma | | | | | |
|---|---|---|---|---|---|---|---|---|---|
| | sizes (number | | | sizes (number | | | (number of | | |
| | of mice and % total) | | | of mice and % total) | | | mice and % total) | | |
| | 5 mm | 3 mm | 2 mm | 5 mm | 3 mm | 2 mm | S | H | B |
| Skp2^{+/+} | 5 | 14 | 2 | 3 | 7 | 5 | 22 | 19 | 16 |
| (n = 44) | 11.4% | 31.8% | 4.5% | 6.82% | 15.9% | 11.4% | 50% | 43.2% | 36.4% |
| Skp2^{-/-} | 5 | 11 | 6 | 4 | 7 | 6 | 15 | 16 | 14 |
| (n = 35) | 14.3% | 31.4% | 17.1% | 11.4% | 20% | 17.1% | 42.9% | 45.7% | 40% |

S—Splenomegaly;
H—Hepatomegaly;
B—both Splenomegaly and Hepatomegaly

Since spontaneous tumorigenesis in Rb1^{+/-} mice requires the loss of the remaining Rb1 allele, it was possible that Skp2 inactivation prevented the second Rb1 mutation, rather than the growth of Rb1-deficient tumors. POMC-Cre and loxP-directed tissue-specific Rb1 deletions were used to artificially generate Rb1^{-/-} pituitary IL melanotrophs (13). To determine whether Skp2 inactivation affects the efficiency of POMC-Cre-loxP-mediated recombination, POMC-Cre;Rosa26R;Skp2^{+/+} and POMC-Cre;Rosa26R;Skp2^{-/-} mice were generated. The POMC-Cre strain induced Cre-loxP-mediated deletion in most of the IL melanotrophs in both Skp2^{+/+} and Skp2^{-/-} mice (FIG. 1A). Since the POMC promoter is also active in corticotrophs in the anterior lobe (AL), scattered AL recombination events were detected in both strains of mice as well.

POMC-Cre;Rb1^{lox/lox}Skp2^{+/+} and POMC-Cre;Rb1^{lox/lox}Skp2^{-/-} mice were then generated and their pituitary glands were examined at 7 weeks of age. As expected (13), POMC-Cre;Rb1^{lox/lox}Skp2^{+/+} mice contained dysplastic nodular lesions across the entire ILs. Unexpectedly, POMC-Cre;Rb1^{lox/lox}Skp2^{-/-} mice did not contain normal-appearing ILs as predicted based on the lack of pituitary tumorigenesis in Rb1^{+/-}Skp2^{-/-} mice. Rather, the ILs of these mice were essentially absent with only a single layer of lining cells separating the anterior and posterior lobes. The ILs of POMC-Cre;Rb1^{lox/lox}Skp2^{+/-} mice were also significantly thinner than normal. These results confirm that Skp2 inactivation blocks tumorigenesis and demonstrate that this effect was achieved not by reverting Rb1-deficient melanotrophs to normal cells, but by eliminating them.

The fate of Rb1 and Skp2 doubly deficient melanotrophs were traced by generating POMC-Cre;Rosa26R;Rb1^{lox/lox}Skp2^{+/+} and POMC-Cre;Rosa26R;Rb1^{lox/lox}Skp2^{-/-} mice and allowing them to age to 10-13 weeks. As shown, the ILs of POMC-Cre;Rosa26R;Rb1^{lox/lox}Skp2^{+/+} mice, observed with hematoxylin stain and EYFP fluorescence, were in more advanced stages of tumorigenesis than those at 7 weeks, whereas the ILs of POMC-Cre;Rosa26R;Rb1^{lox/lox}Skp2^{-/-} mice remained a single-cell layer. Interestingly, the cells in this layer were EYFP positive, suggesting that this single-cell layer environment could prevent death of Rb1 and Skp2 doubly deficient cells or that these cells escaped Rb1 deletion. Rb1 deletion in corticotrophs induced the presence of more corticotrophs in the AL, and combined deletion of Rb1 and Skp2 dramatically reduced their numbers. This indicates that combined Rb1 and Skp2 deletion can eliminate corticotrophs as well as melanotrophs.

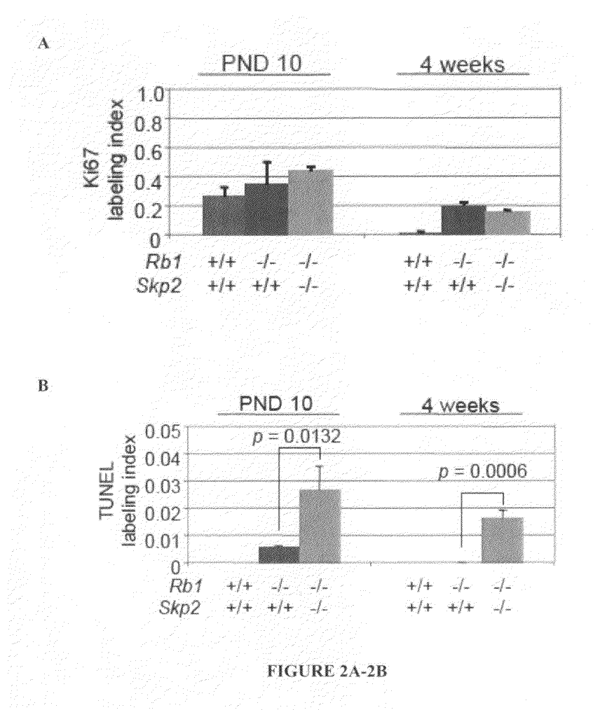

The mice were next harvested at earlier ages to investigate how the ILs were eliminated. At post natal day (PND) 10, the ILs of both POMC-Cre;Rb1^{lox/lox}Skp2^{+/+} and POMC-Cre;Rb1^{lox/lox}Skp2^{-/-} mice showed slightly higher cellularity compared with that of Rb1^{lox/lox} mice. Expression of PCNA (an E2F target gene) and Ki67 (a proliferation marker) were readily observed in melanotrophs indicating the proliferative status of these cells at this age (FIG. 2A). Deletion of Rb1 increased PCNA and Ki67 expression, consistent with deregulation of E2F and proliferation caused by pRb inactivation (FIG. 2A). Skp2 inactivation did not reduce PCNA expression nor the aberrant proliferation of the Rb1-deficient cells (FIG. 2A), but significantly increased TUNEL positive IL cells compared to Rb1^{lox/lox} and POMC-Cre;Rb1^{lox/lox}Skp2^{+/+} controls (FIG. 2B).

At 4 weeks of age, the cells in the ILs of POMC-Cre;Rb 1^{lox/lox}Skp2^{-/-} mice maintained deregulated PCNA expression and proliferation and increased apoptosis (FIG. 2A, 2B). While the aberrantly proliferating ILs of 4 week old POMC-Cre;Rb1^{lox/lox}Skp2^{+/+} mice had become more than 2-fold thicker than that of the Rb1^{lox/lox} controls, the proliferating yet apoptotic ILs of 4-week old POMC-Cre;Rb1^{lox/lox}Skp2^{-/-} mice had become more than 2-fold thinner than normal. Together, these findings indicate that Skp2 is required for the survival of aberrantly proliferating Rb1-deficient melanotrophs and that melanotroph apoptosis caused the elimination of the ILs in POMC-Cre;Rb1^{-/-}Skp2^{-/-} mice.

POMC-Cre;Rb1^{lox/lox} mice allowed the evaluation of the effect of Skp2 on p27 expression during melanotroph tumorigenesis using immunohistochemical staining (IHC). Melanotrophs of Rb1^{lox/lox}, POMC-Cre;Rb1^{lox/lox}, and POMC-Cre;Rb1^{lox/lox}Skp2^{-/-} mice at PND 10 had comparable nuclear p27 protein stains. However, by 4-weeks, p27 levels clearly decreased in melanotrophs of POMC-Cre;Rb1^{lox/lox}Skp2^{-/-} mice, but were maintained in the melanotrophs of POMC-Cre;Rb1^{lox/lox}Skp2^{-/-} mice, suggesting that Skp2 is required for the down regulation of p27 during melanotroph tumorigenesis following Rb1 deletion.

The mechanics of how Skp2 inactivation leads to the failure of p27 downregulation and whether this failure was responsible for the tumor blocking effects of Skp2 inactivation was next investigated. In vitro studies have established that Skp2 mediates p27 ubiquitination in the SCF^{Skp2} ubiquitin ligase after p27 is phosphorylated on T187. However, the in vivo role of this Skp2 function has remained unclear due to divergent findings from Skp2 KO mice (in which all Skp2 functions are absent) and p27T187A KI mice (in which only Skp2's ability to mediate ubiquitination of T187-phosphorylated p27 is absent). Skp2 KO mice showed p27 protein accumulation in certain tissues and smaller body sizes (14), but p27T187A KI mice did not show p27 protein accumulation nor phenocopied Skp2 KO mice (15). Thus, in vivo, Skp2's ability to mediate ubiquitination of T187-phosphorylated p27 does not play a significant role in its ability to regulate p27. Previous findings that pRb inhibits Skp2-mediated p27 ubiquitination by interfering with Skp2 binding to T187-phosphorylated p27 (7) suggests that this Skp2 function may be deregulated and contribute to p27 protein reduction and tumorigenesis following Rb1 loss. To evaluate this prediction, POMC-Cre,Rb1^{lox/lox}p27^{T187A/T187}A and the control Rb1^{lox/lox}p27^{T187A/T187A} mice were generated and their pituitary ILs were examined at 4, 7, and 11 weeks of age.

The ILs of Rb1^{lox/lox}p27^{T187A/T187A} mice appeared normal, consistent with the general lack of abnormality in p27^{T187A/T187A} mice. Following POMC-Cre mediated Rb1 deletion, ILs of POMC-Cre,Rb1^{lox/lox}p27^{T187A/T187A} mice at 4 weeks of age did not show the hyperplastic thickening observed in POMC-Cre,Rb1^{lox/lox} mice but, rather, contained regional thinning. The thinning of the IL became more wide-spread by 7 weeks of age, and by the age of 11 weeks the entire ILs were only 2-3 cell layers thick. The nature of the T187A KI mutation (blocking T187 phosphorylation-dependent ubiquitination of p27 by SCF^{Skp2}) predicted that the tumor blocking effects observed in p27^{T187A/T187A} homozygous mice should also occur in p27^{T187A/+} heterozygous mice, though potentially to a smaller extent. Results confirm this.

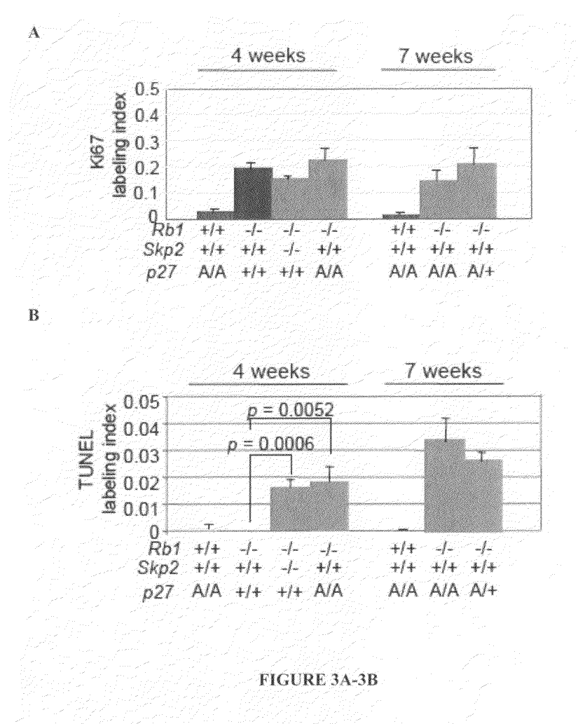

Similar to the effects of Skp2 KO in Rb1-deficient melanotrophs, p27^{T187A} KI did not reduce the deregulated expression of PCNA and proliferation (FIG. 3A), but increased apoptosis (FIG. 3B). These effects were also observed in the presence of one allele of p27^{T187A} (FIG. 3A, 3B). Finally, the reduced p27 expression in melanotrophs in 4-week old POMC-Cre,Rb1^{lox/lox} mice did not occur in melanotrophs in either 4-week or 7-week old POMC-Cre,Rb1^{lox/lox}p27^{T187A/T187A} mice nor in 7-week old POMC-Cre,Rb1^{lox/lox}p27^{T187A/+} mice. This suggests that the T187 phosphorylation-dependent ubiquitination of p27 in the SCF^{Skp2} ubiquitin ligase underlies Skp2's essential role in pituitary tumorigenesis following Rb1 loss, and that the apoptotic ablation of melanotrophs in POMC-Cre;Rb1^{lox/lox}Skp2^{-/-} mice could be explained by a proapoptotic effect of p27 in these cells (16).

Notably, p27T187A KI is not equivalent to Skp2 KO because the ILs of POMC-Cre,Rb1^{lox/lox}xSkp2^{-/-} mice thinned to a greater degree and with faster kinetics than those in POMC-Cre,Rb1^{lox/lox}p27^{T187A/T187A} mice. Skp2 has a growing list of potential substrates in addition to p27, and can support cancer cell survival by protecting cyclin A from inhibition by p27 and p21 (17), and by blocking p53 activation by p300 (18).

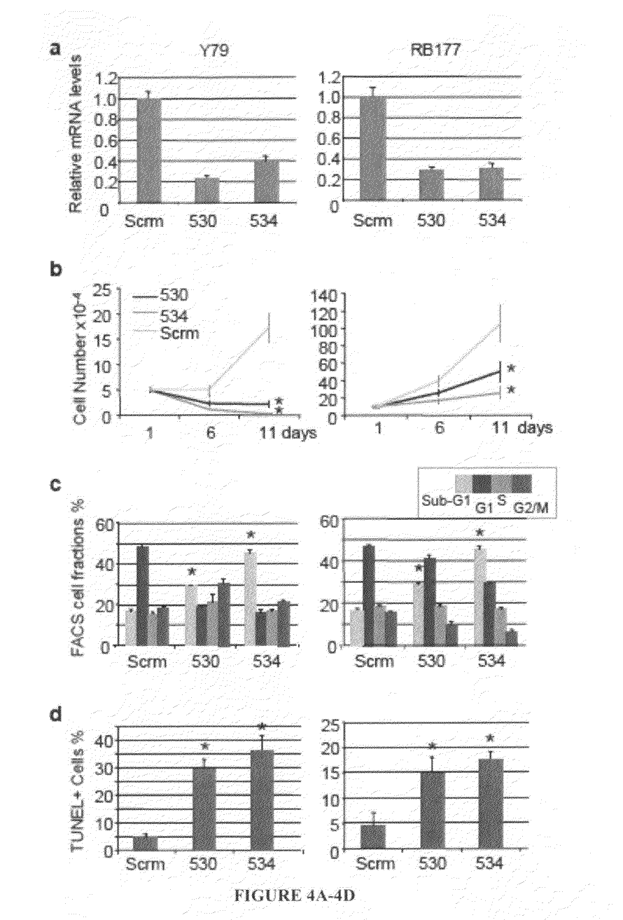

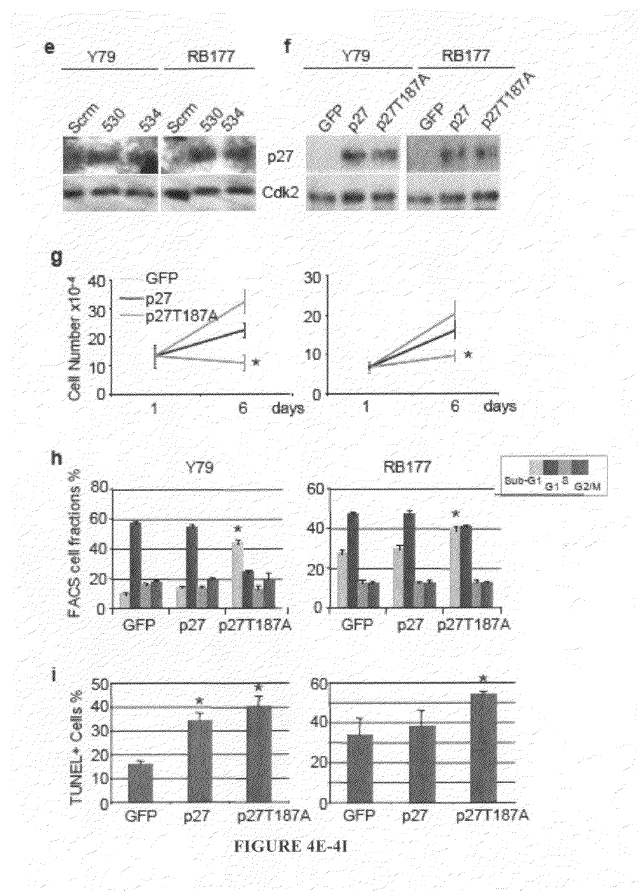

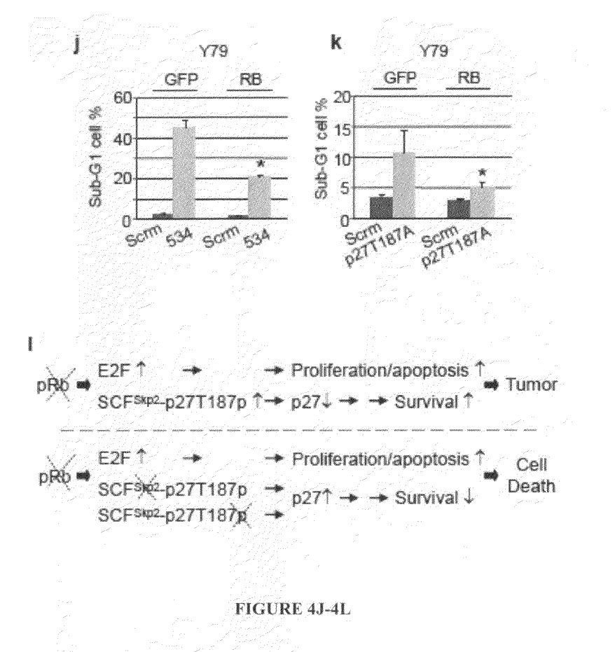

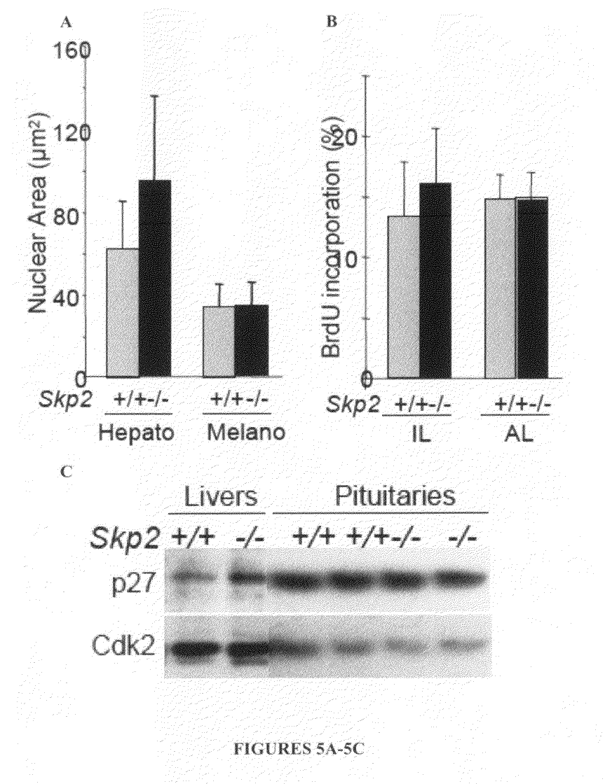

It was next investigated whether the survival function of Skp2 revealed with mouse models was applicable to human tumors that develop due to Rb1 mutations. As retinoblastoma is the main tumor that develops due to Rb1-deficiency in humans, the effect of Skp2 knockdown in retinoblastoma cells was examined. Knockdown of Skp2 (FIG. 4A) significantly inhibited retinoblastoma cell proliferation (FIG. 4B). Skp2 knockdown induced apoptosis, as measured by sub-G1 DNA content and TUNEL staining, but did not diminish S phase population, as measured by FACS (FIG. 4C, 4D). The apoptotic effects of Skp2 knockdown were evident both in the established Y79 cell line and in early passage RB177 cells.

Skp2 knockdown induced accumulation of p27 in human retinoblastoma cells (FIG. 4E). Moreover, ectopic expression of p27 was able to inhibit proliferation and induce apoptosis (FIG. 4F-4I) similar to the effects of Skp2 knockdown. Importantly, the mutant p27T187A was significantly more potent in inhibiting proliferation and inducing apoptosis, consistent with the findings from p27T187A KI mice. Restoration of pRb function largely prevented apoptosis induced either by Skp2 knockdown or by ectopic p27 expression (FIG. 4J, 4K), despite that the modest pRb levels slowed but did not entirely block cell proliferation, suggesting that lack of pRb rendered the retinoblastoma cells dependent on Skp2 and sensitive to aberrantly expressed p27.

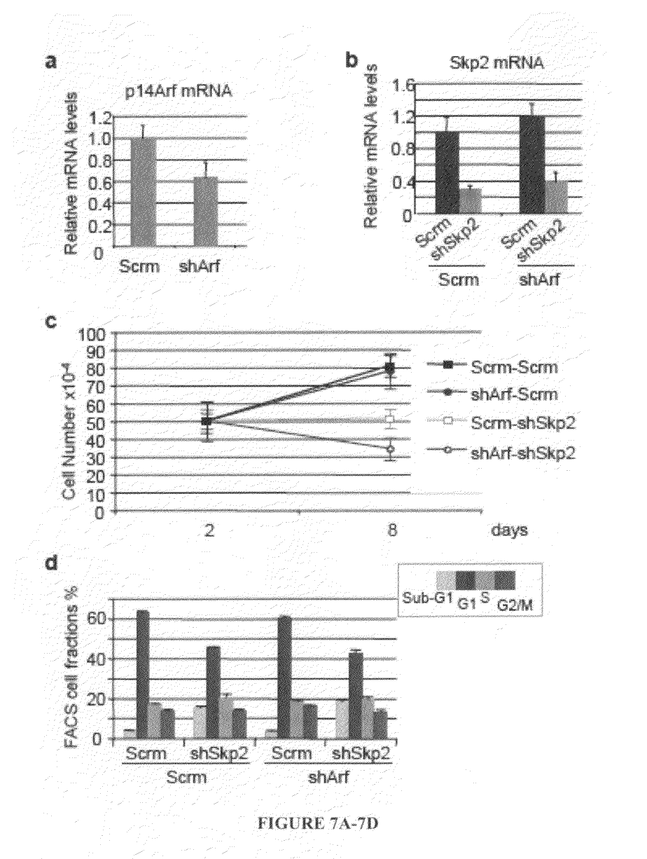

It has been shown that MDM2 plays essential roles for proliferation and survival of retinoblastoma cells and that knockdown of p14Arf diminished the requirement for MDM2 (19). In similar experiments, it was found that knockdown of p14Arf did not mitigate the effects of Skp2 knockdown, suggesting that p14Arf is not a critical target of Skp2 in these cells (FIG. 7).

### 3. Discussion

This invention identifies a normally dispensable mechanism as essential for cell survival following loss of the tumor suppressor pRb. Loss of pRb induces tumorigenesis with full-penetrance in susceptible tissues. Phosphorylation of Thr187 of p27 makes p27 a ubiquitination substrate of the SCF(Skp2) ubiquitin ligase. Inactivation of this T187 phosphorylation dependent ubiquitination of p27, for example, by changing T187 to A187 in the mouse did not show harmful effects. Loss of p27T187 phosphorylation is synthetic lethal with loss of pRb for susceptible cells, identifying a potent preventive and therapeutic target for pRb deficient tumors that is inconsequential to normal cells. Additionally, while loss of p27T187 phosphorylation by itself is harmless to normal cells, it is lethal to cells when combined with pRb loss.

Previous studies have identified many pRb targets and have determined the effects of inactivating some of these pRb targets on tumorigenesis induced by pRb loss using similar mouse models as used in this work. To date, previous studies have only achieved limited successes. Herein, however, the extent of inhibition of tumorigenesis following pRb loss is complete inhibition versus the partial delay of previous studies. Also, the extent of side-effects is better, i.e. innocuous effects on normal cells versus significant toxicity up to embryo lethality by some of the previous studies. This study also differs from existing studies in a fundamental way. Previous studies showed some reversion of pRb deficient tumor cells back to normal-like cells, while this study showed elimination of pRb deficient cells, which provides the basis for the complete inhibition of tumorigenesis following pRb loss by this invention.

Prior to the present invention, inactivation of previously studied pRb targets delayed tumorigenesis in Rb1^{+/-} mice accompanied by reduced tumor cell proliferation (1, 2, 3, 4, 5, 6). In contrast, the present invention reveals that inactivation of Skp2 does not reduce deregulated proliferation of Rb1^{-/-} cells but induces apoptosis, which completely prevents tumorigenesis. This adds a survival arm to the pRb/E2F model of pRb function, in which pRb loss not only deregulates E2F to result in aberrant proliferation and apoptosis through various E2F target genes but also deregulates the SCF^{Skp2}-p27T187p p27 ubiquitination mechanism to down regulate p27 to provide survival support for the aberrantly proliferating pRb-deficient cells (FIG. 4L). When this mechanism is disrupted, either by inactivation of Skp2 or by blocking p27 T187 phosphorylation, the outcome of pRb loss becomes cell death, revealing that Rb1 and Skp2 mutations are synthetically lethal to susceptible cells. Thus, Skp2 is a potentially effective drug target to prevent and treat pRb-deficient tumors. The present invention shows that the p27T187 phosphorylation-dependent function of Skp2 is required for tumorigenesis following pRb loss, yet is not needed for normal development (15), therapeutic targeting of Skp2 can focus on the p27T187-dependent function of Skp2 or p27 T187 phosphorylation.

Inactivation of pRb target Skp2 (7, 8) completely prevents spontaneous tumorigenesis in Rb1^{+/-} mice. Targeted Rb1 deletion in melanotrophs ablates the entire pituitary intermediate lobe when Skp2 is inactivated. Skp2 inactivation does not inhibit aberrant proliferation of Rb1-deleted melanotrophs, but induces their apoptotic death. Eliminating p27 phosphorylation on T187 in p27T187A knockin mice reproduces the effects of Skp2 knockout, identifying p27 ubiquitination by SCF^{Skp}2 ubiquitin ligase as the underlying mechanism for Skp2's essential tumorigenic role in this setting. RB1-deficient human retinoblastoma cells also undergo apoptosis after Skp2 knockdown; and ectopic expression of p27, especially the p27T187A mutant, induces apoptosis. This reveals that Skp2 becomes an essential survival gene when susceptible cells incur Rb1 deficiency.

Skp2 binds T187-phosphorylated p27 for the SCF^{Skp2} ubiquitin ligase to ubiquitinate p27 (9). pRb binds Skp2 to interfere with this binding and ubiquitination (7). pRb-Skp2 binding also bridges Skp2 to the APC-Cdh1 ubiquitin ligase for Skp2 ubiquitination (8). Since Skp2 is an E2F target (10, 11), pRb could repress Skp2 mRNA expression via E2F. Consistent with the above findings, Rb1^{+/-} mice developed Rb1^{-/-} pituitary tumors that had significantly increased amounts of Skp2 mRNA and protein along with decreased amounts of p27 protein (FIG. 1A, 1B).

### REFERENCES

1. Yamasaki, L. et al. Loss of E2F-1 reduces tumorigenesis and extends the lifespan of Rb1(+/−)mice. Nat. Genet. 18, 360-364 (1998).
2. Ziebold, U., Lee, E. Y., Bronson, R. T. & Lees, J. A. E2F3 loss has opposing effects on different pRB-deficient tumors, resulting in suppression of pituitary tumors but metastasis of medullary thyroid carcinomas. Mol. Cell. Biol. 23, 6542-6552 (2003).
3. Lee, E. Y. et al. E2F4 loss suppresses tumorigenesis in Rb mutant mice. Cancer Cell 2, 463-472 (2002).
4. Lasorella, A., Rothschild, G., Yokota, Y., Russell, R. G. & Iavarone, A. Id2 mediates tumor initiation, proliferation, and angiogenesis in Rb mutant mice. Mol. Cell. Biol. 25, 3563-3574 (2005).
5. Takahashi, C. et al. Nras loss induces metastatic conversion of Rb1-deficient neuroendocrine thyroid tumor. Nat. Genet. 38, 118-123 (2006).
6. Takahashi, C., Contreras, B., Bronson, R. T., Loda, M. & Ewen, M. E. Genetic interaction between Rb and K-ras in the control of differentiation and tumor suppression. Mol. Cell. Biol. 24, 10406-10415 (2004).
7. Ji, P. et al. An Rb-Skp2-p27 pathway mediates acute cell cycle inhibition by Rb and is retained in a partial-penetrance Rb mutant. Mol. Cell 16, 47-58 (2004).
8. Binne, U. K. et al. Retinoblastoma protein and anaphase-promoting complex physically interact and functionally cooperate during cell-cycle exit. Nat. Cell Biol. 9, 225-232 (2007).
9. Frescas, D. & Pagano, M. Deregulated proteolysis by the F-box proteins SKP2 and beta-TrCP: tipping the scales of cancer. Nat. Rev. Cancer 8, 438-449 (2008).
10. Zhang, L. & Wang, C. F-box protein Skp2: a novel transcriptional target of E2F. Oncogene 25, 2615-2627 (2005).
11. Yung, Y., Walker, J. L., Roberts, J. M. & Assoian, R. K. A Skp2 autoinduction loop and restriction point control. J. Cell Biol. 178, 741-747 (2007).
12. Jacks, T. et al. Effects of an Rb mutation in the mouse. Nature 359, 295-300 (1992).
13. Vooijs, M., van der Valk, M., to Riele, H. & Berns, A. Flp-mediated tissue-specific inactivation of the retinoblastoma tumor suppressor gene in the mouse. Oncogene 17, 1-12 (1998).
14. Nakayama, K. et al. Targeted disruption of Skp2 results in accumulation of cyclin E and p27(Kip1), polyploidy and centrosome overduplication. EMBO J. 19, 2069-2081 (2000).
15. Malek, N. P. et al. A mouse knock-in model exposes sequential proteolytic pathways that regulate p27Kip1 in G1 and S phase. Nature 413, 323-327 (2001).
16. Carneiro, C. et al. p27 deficiency desensitizes Rb−/− cells to signals that trigger apoptosis during pituitary tumor development. Oncogene 22, 361-369 (2003).
17. Ji, P., Sun, D., Wang, H., Bauzon, F. & Zhu, L. Disrupting Skp2-cyclin A interaction with a blocking peptide induces selective cancer cell killing. Mol. Cancer Ther. 6, 684-691 (2007).
18. Kitagawa, M., Lee, S. H. & McCormick, F. Skp2 Suppresses p53-Dependent Apoptosis by Inhibiting p300. Mol Cell 29, 217-231 (2008).
19. Xu, X. L. et al. Retinoblastoma has properties of a cone precursor tumor and depends upon cone-specific MDM2 signaling. Cell 137, 1018-1031, (2009).
20. Leung, S. W. et al. A dynamic switch in Rb+/− mediated neuroendocrine tumorigenesis. Oncogene 23, 3296-3307 (2004).
21. Balthasar, N. et al. Leptin receptor signaling in POMC neurons is required for normal body weight homeostasis. Neuron 42, 983-891 (2004).
22. Sage, J., Miller, A. L., Perez-Mancera, P. A., Wysocki, J. M. & Jacks, T. Acute mutation of retinoblastoma gene function is sufficient for cell cycle re-entry. Nature 424, 223-228 (2003).
23. Srinivas, S. et al. Cre reporter strains produced by targeted insertion of EYFP and ECFP into the ROSA26 locus. BMC Dev. Biol. 1, 4 (2001).
24. Timmerbeul, I. et al. Testing the importance of p27 degradation by the SCFskp2 pathway in murine models of lung and colon cancer. Proc. Natl. Acad. Sci. USA 103, 14009-14014 (2006).
25. Cobrinik, D., Francis, R. 0., Abramson, D. H. & Lee, T. C. Rb induces a proliferative arrest and curtails Brn-2 expression in retinoblastoma cells. Mol. Cancer 5, 72, (2006).
26. Sheaff, R. J., Groudine, M., Gordon, M., Roberts, J. M. & Clurman, B. E. Cyclin E-CDK2 is a regulator of p27Kip1. Genes Dev. 11, 1464-1478 (1997).
27. Lin et al., Nature. 2010 March 18; 464(7287): 374-379.

1. A method of treating a tumor in a subject comprising administering to the subject an amount of an agent effective to (1) inhibit S-phase kinase-associated protein 2 (Skp2) in the cells of the tumor in the subject, or (2) inhibit phosphorylation of threonine residue no. 187 of p27 in the cells of the tumor in the subject, so as to thereby treat the tumor in the subject.

2. The method of claim 1, wherein the subject is administered an agent which inhibits Skp2 in the cells of the tumor in the subject.

3. The method of claim 2, wherein the agent is a double-stranded siRNA molecule directed against a nucleic acid encoding Skp2.

4. The method of claim 1, wherein the subject is administered an agent which inhibits phosphorylation of threonine residue no. 187 of p27 in the cells of the tumor.

5. The method of claim 1, wherein the tumor is a tumor comprising retinoblastoma protein (pRb)-deficient cells.

6. The method of claim 1, wherein the tumor is a tumor comprising Rb1 +/− cells and/or Rb1 −/− cells.

7. The method of claim 1, wherein the tumor is a tumor of the subject's retina, bone, lung, prostate, breast, bladder, brain, oesophagus, or liver.

8. A method of identifying an agent as an inhibitor of tumorigenesis in retinoblastoma protein (pRb)-deficient cells, the method comprising contacting a p27 with the agent and a kinase which phosphorylates p27, and quantifying the degree of phosphorylation of threonine residue 187 of the p27 (p27T187) by the kinase,
wherein a decrease in the degree of phosphorylation of the p27 as compared to a control indicates that the agent is an inhibitor of tumorigenesis in pRb-deficient cells, while a lack of decrease of phosphorylation of the p27 as compared to a control indicates that the agent does not inhibit tumorigenesis in pRb-deficient cells.

9. A method of identifying an agent as an inhibitor of tumorigenesis in retinoblastoma protein (pRb)-deficient cells, the method comprising contacting a p27 having a phosphorylated threonine residue 187 (p27T187p) with an S-phase kinase-associated protein 2 (Skp2) complex and the agent, and quantifying the interaction between the Skp2 complex and the p27T187p,
wherein a decrease in Skp2 complex interaction with the p27T187p as compared to a control indicates that the agent is an inhibitor of tumorigenesis in pRb-deficient cells, while a lack of decrease in Skp2 complex interaction with the p27T187p as compared to a control indicates that the agent is not an inhibitor of tumorigenesis in pRb-deficient cells.

10. (canceled)

11. (canceled)

12. The method of claim 8, wherein the kinase is a cyclin-dependent kinase (Cdk).

13. The method of claim 8, wherein the degree of phosphorylation of p27T187 or the degree of Skp2 complex interaction with p27T187p is measured by a binding assay.

14. The method of claim 13, wherein the binding assay is a fluorescence-based binding assay or an immunospecific binding assay.

15. The method of any of claim 1, wherein subject is human.

16. The method of claim 1, wherein p27 is p27Kip1.
